(19) United States
(12) Patent Application Publication
(10) Pub. No.: US 2026/0215430 A1
(43) Pub. Date: Jul. 30, 2026

(54) HERBICIDAL COMPOSITIONS

(71) Applicant: SYNGENTA CROP PROTECTION AG, Basel (CH)

(72) Inventors: Eric PALMER, Greensboro, NC (US); Mark KITT, Greensboro, NC (US)

(73) Assignee: SYNGENTA CROP PROTECTION AG, Basel (CH)

(21) Appl. No.: 19/141,825

(22) PCT Filed: Dec. 21, 2023
(86) PCT No.: PCT/US2023/085472

§ 371 Date: Jun. 20, 2025

Related U.S. Application Data
(60) Provisional application No. 63/476,767, filed on Dec. 22, 2022.

(51) Int. Cl. A01N 57/20; A01N 35/06; A01N 37/22; A01N 43/36; A01P 13/00

(52) U.S. Cl. CPC A01N 57/20; A01N 35/06; A01N 37/22; A01N 43/36; A01P 13/00

(57) ABSTRACT

The present disclosure relates to herbicidal combinations and their use in controlling plants or inhibiting plant growth. In particular, herbicidal combinations L-glufosinate in combination with an HPPD inhibitor.

This application claims the benefit of U.S. Provisional Patent Application Ser. No. 63/476,767, filed Dec. 22, 2022, the contents of which are incorporated by reference herein.

The present invention relates herbicidal combinations and their use in controlling plants or inhibiting plant growth. In particular, herbicidal combinations of the invention comprise a L-glufosinate, in combination with an HPPD inhibitor, e.g., mesotrione and bicyclopyrone.

The object of the present invention is to provide herbicidal mixtures which are highly effective against various weed species (particularly at low dose), and is based on the finding that L-glufosinate, in combination with an HPPD inhibitor are particularly efficacious at mediating such weed control.

Thus in a first aspect of the invention, there is provided a composition comprising as component (A) L-glufosinate and as component (B), an HPPD inhibitor, and an agrochemically acceptable salts thereof.

In a second aspect, the invention provides the use of a composition of the invention as a herbicide.

In a third aspect, the invention provides methods of (i) inhibiting plant growth, and (ii) controlling plants, said methods comprising applying to the plants or to the locus thereof: (A) L-glufosinate and (B) an HPPD inhibitor, and agrochemically acceptable salts thereof.

In a fourth aspect, the invention provides a method of controlling grasses and/or weeds in crops of useful plants which comprises applying to the useful plants or locus thereof or to the area of cultivation a herbicidally effective amount of (A) L-glufosinate and (B) an HPPD inhibitor, and agrochemically acceptable salts thereof.

Before certain embodiments are described in greater detail, it is to be understood that this disclosure is not limited to embodiments described, as such may, of course, vary. It is also to be understood that the terminology used herein is for the purpose of describing certain embodiments only, and is not intended to be limiting, since the scope of the present disclosure will be limited only by the appended claims.

Described herein are several definitions. Such definitions are meant to encompass grammatical equivalents.

As used herein, the term “about” when referring to a measurable value such as an amount, a temporal duration, and the like, is meant to encompass variations. Such variations, however, are dependent on the specific component referred to and the context as understood by a person of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Methods and materials similar or equivalent to those described herein can also be used in the practice or testing of the present disclosure; representative illustrative methods, and materials are now described.

Each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

When active ingredients are combined, the activity to be expected (E) for any given active ingredient combination obeys the so-called Colby Formula and can be calculated as follows (Colby, S. R., Calculating synergistic and antagonistic responses of herbicide combination, Weeds, Vol. 15, pages 20-22; 1967):
ppm=milligrams of active ingredient (a.i.) per liter
X=% action by first active ingredient using p ppm of the active ingredient
Y=% action by second active ingredient using q ppm of the active ingredient.

According to Colby, the expected action of active ingredients A+B using p+q ppm of active ingredient is represented by the following formula:

$$E = X + Y - \frac{X \cdot Y}{100}$$

If the action actually observed (O) is greater than the expected action E then the action of the combination is super-additive, i.e. there is a synergistic effect. In mathematical terms, synergism corresponds to a positive value for the difference of (O−E). In the case of purely complementary addition of activities (expected activity), said difference (O−E) is zero. A negative value of said difference (O−E) signals a loss of activity compared to the expected activity.

L-glufosinate and mesotrione, are effective herbicidal compounds, as shown herein.

Accordingly, the combination of the present invention takes advantage of any additive herbicidal activity, and certain embodiments may even exhibit a synergistic effect. This occurs whenever the action of an active ingredient combination is greater than the sum of the actions of the individual components.

Combinations of the invention may also provide for an extended spectrum of activity in comparison to that obtained by each individual component, and/or permit the use of lower rates of the individual components when used in combination to that when used alone, in order to mediate effective herbicidal activity.

In addition, it is also possible that the composition of the invention may show increased crop tolerance, when compared with the effect of the component (A) alone. This occurs when the action of an active ingredient combination is less damaging to a useful crop than the action of one of the active ingredients alone. As stated above, compositions of the invention comprise as component (A) L-glufosinate, or an agrochemically acceptable salt thereof.

Glufosinate,

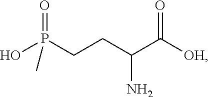

also called phosphinothricin, is often sold as an ammonium salt. Glufosinate is a naturally occuring broad-spectrum herbicide produced by several species of Streptomyces soil bacteri. Glufosinate is a group 10 herbicde and inhibits glutamine synthetase. Reports of glufosinate resistant Italian ryegrass, Lolium perenne ssp. Multiflorum, have been reported in Oregon and California. (Heap, I. The International Herbicide-Resistant Weed Database. Accessed Mar. 30, 2020.) have been found. L-glufosinate is the L-isomer of glufosinate,

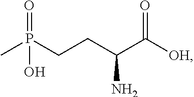

and is more active than the D-isomer.

Mesotrione,

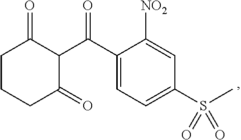 [SMILES: CS(=O)(=O)c1ccc(C(=O)C2C(=O)CCCC2=O)c([N+](=O)[O-])c1]

is a Group 27 herbicide. Group 27 herbicides are bleaching herbicides, and inhibit 4-hydroxyphenyl-pyruvatedioxygenase (4-HPPD). Mesotrione is specifically triketone group 27 compound. In certain embodiments, mesotrione can be chelated by iron or copper, preferably copper.

Currently Group 27 herbicides can be classified as triketone compounds, (e.g., mesotrione and sulcotrione) isoxazole compounds, (e.g., isoxachlortole and isoxaflutole) pyrazole compounds (e.g. benzofenap, pyrazolynate, and pyrazoxyfen). Other classified Group 27 herbicides include: benzobicyclon, bromobutide (chloro)-flurenol, cinmethylin, cumyluron, dazomet, dymron, etobenzanid, fosamine, indanofan, metam, oxaziclomefone, oleic acid, pelargonic acid, and pyributicarb.

Certain embodiments of the disclosure include, glufosinate and mesotrione. In some embodiments, synergy may be present with glufosinate and mesotrione. In typical embodiments, additional glufosinate or mesotrione may be needed to show synergistic activity as compared to L-glufosinate and mesotrione.

Some embodiments further include a chloroacetamide herbicide or a Group 15 herbicide. Group 15 herbicides are growth inhibitors which are thought to inhibit very long chain fatty acid (VLCFA) synthesis. Chemical classes associated with Group 15 herbicides are chloroacetamides, oxyacetamides, and pyrazoles. Examples of Group 15 herbicides include acetochlor, alachlor, dimethenamid, metolachlor, flufenacet and pyroxasulfone. In preferred embodiments, S-metolachlor is used.

Throughout this document the expression “composition” should be interpreted as meaning the various mixtures or combinations of components (A) and (B), for example in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the components (A) and (B) is not essential for working the present invention.

The term “herbicide” as used herein means a compound that controls or modifies the growth of plants. The term “herbicidally effective amount” means the quantity of such a compound or combination of such compounds that is capable of producing a controlling or modifying effect on the growth of plants. Controlling or modifying effects include all deviation from natural development, for example killing, retardation, leaf burn, albinism, dwarfing and the like.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” denotes all generative parts of a plant, for example seeds or vegetative parts of plants such as cuttings and tubers. It includes seeds in the strict sense, as well as roots, fruits, tubers, bulbs, rhizomes, and parts of plants.

The term “safener” as used herein means a chemical that when used in combination with a herbicide reduces the undesirable effects of the herbicide on non-target organisms, for example, a safener protects crops from injury by herbicides but does not prevent the herbicide from killing the weeds.

Crops of useful plants in which the composition according to the invention can be used include perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

Crops are to be understood as being those which are naturally occurring, obtained by conventional methods of breeding, or obtained by genetic engineering. They include crops which contain so-called output traits (e.g. improved storage stability, higher nutritional value and improved flavour).

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. Other examples include crops that have been rendered tolerant to HPPD-inhibitors, for example HPPD tolerant soybeans, that have been modified to express a modified p-hydroxyphenylpyruvate dioxygenase (hppd) enzymes. Such enzymes may be derived from a variety of sources. In one example, the gene source is Pseudomonas fluorescens, e.g. strain A32, and the gene is hppdPF W336, for example, as found in Event FG72. In another example, the HPPD tolerant soybean plant includes a plant having a reduced expression of at least one 4-hydroxyphenylpyruvate reductase (HPPR) enzyme. In other examples, HPPD tolerant soybeans include soybeans modified to overexpress a gene coding for a tolerant HPPD, for example derived from Avena sativa (see for example US2011/0173718) or Arabidopsis (e.g. as in WO2013/064964, WO2014/177999). In other examples, HPPD tolerant soybeans include soybeans having a modified expression of triketone dioxygenase (TDO) proteins, for example, as found in soybean plants containing event MON 94313.

Crop plants can also be tolerant to protoporphyrinogen oxidase (PPO) inhibitors. Such plants can be obtained, for example, by transforming crop plants with nucleic acids which encode a suitable protoporphyrinogen oxidase, which may contain a mutation in order to make it more resistant to the PPO inhibitor. Examples of such nucleic acids and crop plants are disclosed in WO95/34659, WO97/32011, WO2012/080975, WO2013/189984, WO2015/022636, WO2015/022640, WO2015/092706, WO2016/099153, WO2017/023778, WO2017/039969, WO2017/217793, WO2017/217794, WO2018/114759, WO2019/117578, WO2019/117579 and WO2019/118726, all incorporated by reference in their entirety herein.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Further examples of such crops include: RR1, RR2 XTEND, XtendFlex, LibertyLink, Enlist E3, GTS 40-3-2, MON89788, A2704-12, GT27, LLGT27, or GMB151. In preferred many embodiments, the crops contain the MON94313 event or the MON87429 event.

In aspects of the invention, in any particular embodiment, the weeds, e.g. to be controlled and/or growth-inhibited, may be monocotyledonous or dicotyledonous weeds, which are tolerant or resistant to one or more other herbicides for example, HPPD inhibitor herbicides such as mesotrione.

Compositions of this invention can also be mixed with one or more further pesticides including herbicides typically different to HPPD-inhibitors and HPPD-inhibitors, for example, fungicides, insecticides, nematocides, bactericides, acaricides, growth regulators, chemosterilants, semiochemicals, repellents, attractants, pheromones, feeding stimulants or other biologically active compounds to form a multi-component pesticide giving an even broader spectrum of agricultural protection.

Similarly compositions of the invention (which includes those comprising one or more additional pesticide as described in the preceding paragraph) can further include one or more safeners. In particular, the following safeners are especially preferred: AD 67 (MON 4660), benoxacor, cloquintocet-mexyl, cyometrinil, cyprosulfamide, dichlormid, dicyclonon, dietholate, fenchlorazole-ethyl, fenclorim, flurazole, fluxofenim, furilazole, furilazome, isoxadifen-ethyl, mefenpyr-diethyl, mephenate, oxabetrinil, naphthalic anhydride (CAS RN 81-84-5), TI-35, N-isopropyl-4-(2-methoxy-benzoylsulfamoyl)-benzamide (CAS RN 221668-34-4) and N-(2-methoxybenzoyl)-4-[(methylaminocarbonyl)amino]benzenesulfonamide. Such safeners may also be used in the form of esters or salts, as mentioned e.g. in The Pesticide Manual, 15th Ed. (BCPC), 2009. Thus, the reference to cloquintocet-mexyl also applies to cloquintocet and to a lithium, sodium, potassium, calcium, magnesium, aluminium, iron, ammonium, quaternary ammonium, sulfonium or phosphonium salt thereof as disclosed in WO02/34048 and the reference to fenchlorazole-ethyl also applies to fenchlorazole, etc.

The compositions of the invention can be applied before or after planting of the crops, before weeds emerge (pre-emergence application) or after weeds emerge (post-emergence application). Where a safener is combined with mixtures of the invention, it is preferred that the mixing ratio of component (A) or component (B) herbicide to safener is from 100:1 to 1:10, especially from 20:1 to 1:1.

It is possible that the safener and the compositions of the invention are applied simultaneously. For example, the safener and the composition of the invention might be applied to the locus pre-emergence or might be applied to the crop post-emergence. It is also possible that the safener and the composition of the invention are applied sequentially. For example, the safener might be applied before sowing the seeds as a seed treatment and the composition of the invention might be applied to the locus pre-emergence or might be applied to the crop post-emergence.

However, the skilled man will appreciate that compositions of the invention are particularly useful in non-selective burn-down applications, and as such may also be used to control volunteer or escape crop plants. In such situations, it is clearly not necessary to include a safener in a composition of the invention.

In general, the mixing ratio (by weight) of component (A) herbicide to the compound of component (B) is from 0.01:1 to 100:1 (A:B), more preferably from 0.05:1 to 20:1 (A:B), even more preferably from about 10:1 to about 1:10. Accordingly, preferred ratios—and range composed thereof—include 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, and 10:1.

When applied in a composition of the invention component (A) is typically applied at a rate of 25 to 2000 g ha, more particularly 25, 50, 75, 100, 125, 150, 200, 250, 300, 400, 500, 750, 800, 1000, 1250, 1500, 1800, or 2000 g/ha, and rates between any of such numbers. Such rates of component (A) are applied typically in association with 5 to 2000 g/ha of component (B), and more specifically in association with 5, 10, 15, 20, 25, 50, 75, 100, 120, 125, 140, 150, 200, 240, 250, 300, 400, 480, 500, 750, 1000, 1250, 1500, 1800, or 2000 g/ha of component (B), and rates between any of such numbers. The Examples described herein illustrate but do not limit the range of rates of components (A) and (B) that may be employed in the invention.

The amount of a composition according to the invention to be applied, will depend on various factors, such as the compounds employed; the subject of the treatment, such as, for example plants, soil or seeds; the type of treatment, such as, for example spraying, dusting or seed dressing; or the application time. In agricultural practice the application rates of the composition according to the invention depend on the type of effect desired, and typically range from 30 to 4000 g of total composition per hectare, and more commonly between 30 and 2000 g/ha. The application is generally made by spraying the composition, typically by tractor mounted sprayer for large areas, but other methods such as dusting (for powders), drip or drench can also be used.

The compositions of the invention can advantageously be used in the below-mentioned formulations (in which case “active ingredient” relates to the respective mixture of component (A) with a compound of component (B) or, when a safener is also used, the respective mixture of component (A) with the compound of component (B) and the safener).

The individual components of the composition of the invention may be utilised as the technical active ingredient as produced. More typically however, the compositions according to the invention may be formulated in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The formulations according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The formulations generally comprise from 0.1 to 99% by weight, especially from 0.1 to 95% by weight, of compounds (A) and (B) and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %), wherein the term “active ingredient” refers to the total weight % of the combination of all active ingredients in the composition:

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

Various aspects and embodiments of the present invention will now be illustrated in more detail by way of example. It will be appreciated that modification of detail may be made without departing from the scope of the invention.

### BIOLOGICAL EXAMPLES

In a postemergence field study on a natural weed populations. Herbicides were applied to 4-5″ weeds and efficacy was evaluated at 14 and 28-35 days after application. Rates were in g ai/ha. Data shown was small seeded broadleaf (SSBL; AMPA=Amaranthus palmeri; CHEAL=Chenopodium Album L.; AMBEL=Ambrosia Artemisiifolia L.) control 28-31 DAA. 100% was completely dead and 0% was no plant response.

| | | | | SSBL |
|---|---|---|---|---|
| | | | | (AMAPA, |
| Active | Application | | | CHEAL, |
| Ingredient(s) | rate (g/ha) | All weeds | Grasses | AMBEL) |
| Mesotrione | 105 | 44 | 35 | 26 |
| L-glufosinate | 328 | 70 | 83 | 55 |
| Mesotrione + | 105 + | 83 | 89 | 67 |
| L-glufosinate | 328 | | | |
| EXPECTED | | | | |
| Mesotrione + | 105 + | 86 | 85 | 83 |
| L-glufosinate | 328 | | | |
| OBSERVED | | | | |

As observed, the combination of mesotrione and L-glufosinate provided greater than expected, synergistic control of SSBL, and even all weeds, as calculated by Colby.

In a post-emergence study of AMPA, CHEAL, and ABUTH (Velvet Leaf (Abutilon theophrast)) applications were done to 2 to 4-inch weeds with a spray volume of 15 gallons/acre. Treatments applied are described below, and further included an adjuvant.

| Trt # | Herbicide 1 | Herbicide 2 | Rates (g ai/ha) |
|---|---|---|---|
| 1 | Check | | |
| 2 | L-glufosinate | | 5 |
| 3 | L-glufosinate | | 10 |
| 4 | L-glufosinate | | 30 |
| 5 | L-glufosinate | | 60 |
| 6 | L-glufosinate | | 120 |
| 7 | | Mesotrione | 1.5 |
| 8 | | Mesotrione | 3 |
| 9 | | Mesotrione | 6 |
| 10 | | Mesotrione | 12 |
| 11 | | Mesotrione | 24 |
| 12 | | Mesotrione | 60 |
| 13 | L-glufosinate | Mesotrione | 30 + 12 |
| 14 | L-glufosinate | Mesotrione | 30 + 6 |
| 15 | L-glufosinate | Mesotrione | 30 + 3 |
| 16 | L-glufosinate | Mesotrione | 30 + 1.5 |
| 17 | L-glufosinate | Mesotrione | 60 + 12 |
| 18 | L-glufosinate | Mesotrione | 60 + 6 |
| 19 | L-glufosinate | Mesotrione | 60 + 3 |
| 20 | L-glufosinate | Mesotrione | 60 + 1.5 |
| 21 | L-glufosinate | Mesotrione | 120 + 6 |
| 22 | L-glufosinate | Mesotrione | 120 + 3 |
| 23 | L-glufosinate | Mesotrione | 10 + 1.5 |
| 24 | L-glufosinate | Mesotrione | 10 + 3 |
| 25 | L-glufosinate | Mesotrione | 10 + 6 |
| 26 | L-glufosinate | Mesotrione | 10 + 12 |
| 27 | L-glufosinate | Mesotrione | 10 + 24 |
| 28 | L-glufosinate | Mesotrione | 10 + 60 |
| 29 | L-glufosinate | Mesotrione | 5 + 60 |

The results of AMAPA control with L-GLU+MST mixtures at 7 days after treatment are summarized in the below matrix, where bolded values represent synergy as calculated by Colby.

L-GLU (g ai/ha)

| | | | | | |
|---|---|---|---|---|---|
| 0 | 5 | 10 | 30 | 60 | 120 |

| | |
|---|---|
| MST(g ai/ha) | % Control |

| | | | | | | |
|---|---|---|---|---|---|---|
| 0 | 0 | 27 | 33 | 83 | 97 | 100 |
| 1.5 | 20 | | 47 | 77 | 93 | |
| 3 | 30 | | 62 | 72 | 97 | 100 |
| 6 | 37 | | 43 | 97 | 90 | 100 |
| 12 | 72 | | 65 | 90 | 98 | |
| 24 | 85 | | 84 | | | |
| 60 | 85 | 93 | 96 | | | |

The results of ABUTH control with L-GLU+MST mixtures at 7 days after treatment are summarized in the below matrix, where bolded values represent synergy as calculated by Colby.

L-GLU(g ai/ha)

| | | | | | |
|---|---|---|---|---|---|
| 0 | 5 | 10 | 30 | 60 | 120 |

| | |
|---|---|
| MST(g ai/ha) | % Control |

| | | | | | | |
|---|---|---|---|---|---|---|
| 0 | 0 | 2 | 8 | 38 | 67 | 87 |
| 1.5 | 85 | | 80 | 87 | 96 | |
| 3 | 83 | | 92 | 88 | 97 | 92 |
| 6 | 95 | | 92 | 100 | 96 | 98 |
| 12 | 92 | | 94 | 95 | 98 | |
| 24 | 96 | | 93 | | | |
| 60 | 92 | 88 | 92 | | | |

The results of CHEAL control with L-GLU+MST mixtures at 7 days after treatment are summarized in the below matrix, where bolded values represent synergy as calculated by Colby.

L-GLU(g ai/ha)

| | | | | | |
|---|---|---|---|---|---|
| 0 | 5 | 10 | 30 | 60 | 120 |

| | |
|---|---|
| MST(g ai/ha) | % Control |

| | | | | | | |
|---|---|---|---|---|---|---|
| 0 | 0 | 3 | 8 | 72 | 90 | 90 |
| 1.5 | 78 | | 67 | 87 | 95 | |
| 3 | 85 | | 85 | 96 | 100 | 97 |
| 6 | 92 | | 89 | 96 | 98 | 100 |
| 12 | 93 | | 92 | 96 | 100 | |
| 24 | 93 | | 93 | | | |
| 60 | 90 | 93 | 94 | | | |

In a post-emergence study of AMPA, CHEAL, and Setaria faberi (SETFA) applications were done to 2 to 4-inch weeds with a spray volume of 15 gallons/acre. Treatments applied are described below, and further included an adjuvant.

| Trt # | Herbicide 1 | Herbicide 2 | Rates (g ai/ha) |
|---|---|---|---|
| 1 | Check | | |
| 2 | L-glufosinate | | 5 |
| 3 | L-glufosinate | | 10 |
| 4 | L-glufosinate | | 30 |
| 5 | L-glufosinate | | 60 |
| 6 | L-glufosinate | | 120 |
| 7 | | Bicyclopyrone | 3.13 |
| 8 | | Bicyclopyrone | 6.25 |
| 9 | | Bicyclopyrone | 12.5 |
| 10 | | Bicyclopyrone | 25 |
| 11 | | Bicyclopyrone | 50 |
| 12 | | Bicyclopyrone | 100 |
| 13 | L-glufosinate | Bicyclopyrone | 5 + 25 |
| 14 | L-glufosinate | Bicyclopyrone | 5 + 50 |
| 15 | L-glufosinate | Bicyclopyrone | 5 + 100 |
| 16 | L-glufosinate | Bicyclopyrone | 10 + 12.5 |
| 17 | L-glufosinate | Bicyclopyrone | 10 + 25 |
| 18 | L-glufosinate | Bicyclopyrone | 10 + 50 |
| 19 | L-glufosinate | Bicyclopyrone | 10 + 100 |
| 20 | L-glufosinate | Bicyclopyrone | 30 + 3.13 |
| 21 | L-glufosinate | Bicyclopyrone | 30 + 6.25 |
| 22 | L-glufosinate | Bicyclopyrone | 30 + 12.5 |
| 23 | L-glufosinate | Bicyclopyrone | 30 + 25 |
| 24 | L-glufosinate | Bicyclopyrone | 30 + 50 |
| 25 | L-glufosinate | Bicyclopyrone | 30 + 100 |
| 26 | L-glufosinate | Bicyclopyrone | 60 + 3.13 |
| 27 | L-glufosinate | Bicyclopyrone | 60 + 6.25 |
| 28 | L-glufosinate | Bicyclopyrone | 60 + 12.5 |
| 29 | L-glufosinate | Bicyclopyrone | 60 + 25 |
| 30 | L-glufosinate | Bicyclopyrone | 60 + 50 |
| 31 | L-glufosinate | Bicyclopyrone | 60 + 100 |
| 32 | L-glufosinate | Bicyclopyrone | 120 + 3.13 |
| 33 | L-glufosinate | Bicyclopyrone | 120 + 6.25 |
| 34 | L-glufosinate | Bicyclopyrone | 120 + 12.5 |

The results of AMAPA control with L-GLU+BIR (bicyclopyrone) mixtures at 9 days after treatment are summarized in the below matrix, where bolded values represent synergy as calculated by Colby.

L-GLU (g ai/ha)

| | | | | | |
|---|---|---|---|---|---|
| 0 | 5 | 10 | 30 | 60 | 120 |

| | |
|---|---|
| BIR(g ai/ha) | % Control |

| | | | | | | |
|---|---|---|---|---|---|---|
| 0 | 0 | 17 | 23 | 57 | 87 | 93 |
| 1.56 | 27 | | 45 | 63 | 90 | 94 |
| 3.13 | 37 | | 40 | 65 | 85 | 100 |
| 6.25 | 48 | 48 | 52 | 63 | 80 | 100 |
| 12.5 | 53 | 50 | 63 | 80 | 85 | |
| 25 | 80 | 77 | 80 | 93 | | |
| 50 | 90 | 80 | 86 | | | |

The results of CHEAL control with L-GLU+BIR (bicyclopyrone) mixtures at 9 days after treatment are summarized in the below matrix, where bolded values represent synergy as calculated by Colby.

L-GLU (g ai/ha)

| | | | | | |
|---|---|---|---|---|---|
| 0 | 5 | 10 | 30 | 60 | 120 |

| | |
|---|---|
| BIR(g ai/ha) | % Control |

| | | | | | | |
|---|---|---|---|---|---|---|
| 0 | 0 | 0 | 13 | 38 | 88 | 100 |
| 1.56 | 58 | | 50 | 63 | 97 | 100 |
| 3.13 | 75 | | 72 | 83 | 98 | 100 |
| 6.25 | 88 | 88 | 93 | 97 | 99 | 98 |
| 12.5 | 92 | 92 | 93 | 100 | 100 | |
| 25 | 96 | 98 | 97 | 100 | | |
| 50 | 97 | 98 | 100 | | | |

The results of SETFA control with L-GLU+BIR (bicyclopyrone) mixtures at 7 and 14 days after treatment, respectively, are summarized in the below matrix, where bolded values represent synergy as calculated by Colby.

7 Days after Treatment

L-GLU (g ai/ha)

| | | | | | |
|---|---|---|---|---|---|
| 0 | 5 | 10 | 30 | 60 | 120 |

| | |
|---|---|
| BIR(g ai/ha) | % Control |

| | | | | | | |
|---|---|---|---|---|---|---|
| 0 | 0 | 13 | 48 | 85 | 95 | 100 |
| 3.13 | 28 | | | 90 | 98 | 100 |
| 6.25 | 35 | | | 91 | 98 | 100 |
| 12.5 | 62 | | 47 | 97 | 99 | 100 |
| 25 | 73 | 17 | 43 | 98 | 99 | |
| 50 | 78 | 78 | 72 | 99 | 100 | |
| 100 | 77 | 80 | 75 | 100 | 100 | |

14 Days after Treatment

L-GLU (g ai/ha)

| | | | | | |
|---|---|---|---|---|---|
| 0 | 5 | 10 | 30 | 60 | 120 |

| | |
|---|---|
| BIR(g ai/ha) | % Control |

| | | | | | | |
|---|---|---|---|---|---|---|
| 0 | 0 | 10 | 35 | 65 | 89 | 100 |
| 3.13 | 23 | | | 68 | 91 | 98 |
| 6.25 | 32 | | | 77 | 93 | 100 |
| 12.5 | 45 | | 38 | 88 | 95 | 100 |
| 25 | 58 | 12 | 33 | 78 | 78 | |
| 50 | 75 | 67 | 63 | 88 | 96 | |
| 100 | 90 | 78 | 78 | 93 | 100 | |

1. A composition, comprising:
(A) L-glufosinate, or agrochemically acceptable salts thereof; and
(B) an HPPD inhibitor, or agrochemically acceptable salts thereof.

2. The composition of claim 1, wherein (A) and (B) are present in a synergistic weight ratio as defined by the Colby Formula.

3. The composition of claim 1, wherein the weight ratio of (A) to (B) is about 0.01:1 to 100:1 (A:B), more preferably from 0.05:1 to 20:1 (A:B), even more preferably from about 10:1 to about 1:10.

4. The composition of claim 1, wherein the HPPD inhibitor is chelated preferably copper chelated or iron chelated.

5. The composition of claim 1, additionally comprising an agriculturally acceptable formulation adjuvant.

6. The composition of claim 1, further comprising at least one additional pesticide.

7. The herbicidal composition according to claim 8, wherein the additional pesticide is a herbicide or herbicide safener.

8. The composition of claim 1, wherein the HPPD inhibitor is mesotrione.

9. The composition of claim 1, wherein the HPPD inhibitor is bicyclopyrone.

10. The composition of claim 1, wherein the composition further comprises a chloroacetamide herbicide.

11. The composition according to claim 10, wherein the chloroacetamide herbicide is S-metolachlor.

12. A method of controlling unwanted plant growth, comprising: applying the composition of claim 1 to the unwanted plants or to the locus thereof.

13. A method comprising: applying (A) L-glufosinate, or agrochemically acceptable salts thereof; and (B) mesotrione, or agrochemically acceptable salts thereof to the unwanted plants or to the locus thereof sequentially.

14. The method of claim 12, wherein the applying is to a soybean field.

15. The method of claim 14, wherein the soybeans in the soybean field have been rendered tolerant to at least one of an HPPD-inhibitor herbicide and a PPO-inhibitor herbicide.

16. The method of claim 15, wherein the soybeans comprise at least one of an LLGT27 event, a MON94313 event, a GMB1541 event, and an event that expresses a protoporphyrinogen oxidase.

17. The method of claim 12, wherein the applying is to a cotton field.

18. The method of claim 12, wherein the applying is to a corn field.

19. The method of claim 12, wherein the unwanted plants are small seeded broadleaf.

20. The method of claim 19, wherein the unwanted plant is Amaranthus palmeri.

21. The method of claim 19, wherein the unwanted plant is Chenopodium Album.

22. The method of claim 19, wherein the unwanted plant is Ambrosia Artemisiifolia L.
